(19) United States
(12) Patent Application Publication
(10) Pub. No.: US 2006/0106424 A1
(43) Pub. Date: May 18, 2006

(54) Ultrasound device and method of use

(76) Inventor: Max Bachem, Langfeld (DE)

Correspondence Address: CHIEF PATENT COUNSEL;SMITH & NEPHEW, INC., 1450 BROOKS ROAD, MEMPHIS, TN, 38116, US

(21) Appl. No.: 11/220,128

(22) Filed: Sep. 6, 2005

(30) Foreign Application Priority Data
Sep. 4, 2004 (GB) 0419673.9
Feb. 21, 2005 (GB) 0503523.3

(51) Int. Cl. A61N 1/39

(52) U.S. Cl. 607/1

(57) ABSTRACT

A method of treating bacterial and/or fungal infections by stimulating a cell, cells or tissue with ultrasound.

### RELATED APPLICATIONS

This document claims the benefit of the filing dates of GB 0419673.9, filed Sep. 4, 2004 and GB 0503523.3, filed Feb. 21, 2005, the entire contents of both of which are hereby incorporated by this reference.

### RELATED FIELDS

The present invention relates to an Ultrasound Device and its method of use. In particular it relates to using ultrasound to stimulate cells of the body. It also relates to therapeutically treating bacterial infection using ultrasound.

### BACKGROUND

The mechanical energy of ultrasound is transmitted through and into biological tissues as an acoustic pressure wave at frequencies above the limit of human hearing. Ultrasound is in use as a therapeutic, surgical, and diagnostic tool. The biomedical applications of ultrasound result from thermal and non-thermal (mechanical) effects. Ultrasound treatment is a non-invasive therapy for pathological conditions such as inflammation of soft tissue. Low intensity pulsed ultrasound (LIPUS) is often between 30-50 mW/cm², and the acoustic wave is often delivered in a 1 kHz repetition rate and a pulse width of 200 μs. Moreover, this intensity is thought not to cause tissue injury since it is also used in diagnostics. Based on the positive clinical and animal trials, and a host of in vitro studies, the Food and Drug Administration approved the application of LIPUS for the accelerated healing of fresh fractures in the 1994 and the treatment of established bone non- unions in 2000.

Macrophages are essential immunocytes. They act as scavengers to phagocytose and digest pathological organisms, non-functional host cells, bacteria- filled neutrophilis, damaged matrix and foreign debris from the wound area. Furthermore, they also present antigens to T cells, hereby initiating T cell mediated immunoreactions to improve the healing process. Macrophages produce a plethora of biologically advice substances, which include cytokiness, matrix metalloproteinases (MMPS) and extracellular matrix mettalloproteinase inducer (EMMPRIN).

Cytokines are multifunctional signalling proteins that regulate a plethora of cellular activities such as purification of wound area, matrix remodelling, and granulation tissue formation. MMPs are a growing family of metalloendopeptidases that cleave the protein components of extra cellular matrix and play a central role in tissue remodelling. Furthermore, MMPs are implicated in the functional regulation of a host of non-ECM molecules that include cytokines and their receptors, adhesion receptors, and a variety of enzymes. MMPs therefore play an important role in the control of cellular interactions with and response to their environment, which is beneficial to promote tissue turnover. EMMPRIN is an integral plasma membrane glycoprotein of the immunoglobulin super family that probably has several functions, but one established property is its ability to induce the synthesis of various MMPs. Thus EMMPRIN can synergize with MMPs to regulate tissue reconstruction. In summary, through production of the bioactive substances, macrophages orchestrate the complex processes of cellular proliferation and functional tissue regeneration within wounds. Cells reside in the extra cellular matrix network. The cell-ECM contacts are thought not only as sites of original transduction from the ECM, but also as structural links between the ECM and the cytoskeleton. At these contracts, mechanical signals produced by LIPUS may be transmitted into the cells via membrane-coupled mechanosensors that connects ECM to cytoskeleton, which subsequently initiates signalling cascades that are responsible for cell behaviour.

So far, some evidence suggests that the intriguing candidates of the mechanosensors are integrins. Integrins comprise a large family of cell surface receptors, which are composed of two non-covalently associated transmembrane glycoprotein subunits, a and β. Each aβ heterodimer contains a large extracellular domain responsible for ligand binding, a single transmembrane domain and a cytoplasmic domain involved in signal transduction pathway.

Upon ligation with ECM in response to extracellular stimuli, integrins become clustered, and the signals are transmitted to the cytoplasmic domains. Cytoplasmic domain binding proteins include protein tyrosine kinases, focal adhesion kinase (FAK), Syk, and cytoskeletal proteins. Hereby a variety of signalling cascades are initiated, among these, one of the earliest events is tyrosine phosphorylation augmentation of multiple cytoskeletal-associated proteins, such as paxillin, cortactin and p130^{cas}. Accumulating evidence implies that FAK, its related family members (Pyk2), phosphatidylinositol kinase (P13K), Syk, Src-family kinases and mitogen- actived protein kinases (MAPKs) family are the components of integrin-mediated signal transduction.

Considering cell behaviour integrins are involved in many key biological processes, including cell-extracellular matrix adhesion and inflammatory phenomena.

### SUMMARY

Another object of the present invention is to stimulate or enhance the activity of cells of the body that clear bacterial and/or fungal infection. Another object of the present invention is to stimulate or enhance the activity of cells of the body to kill bacteria and/or fungi. Another object of the present invention is to increase macrophage activity.

Yet another object of the present invention is to stimulate or enhance the phagocytic action of phagocytic cells.

Also according to the invention there is provided a method for treating bacterial infections characterised by the step of applying ultrasound to, adjacent, or near the infected site.

Also according to the present invention there is provided a method of treating fungal infections characterised by the step of applying ultrasound to, adjacent, or near the infected site.

The bacterial or fungal infection may be, but not limited to a localised infection on a body but may also be non-localised infections such as septicaemia.

The infected site may be the blood, bone or joint soft tissue, brain, skin, meninges or other.

The ultrasound may be a pulsed radio frequency ultrasound signal. Ideally the pulsed radio frequency signal has a frequency in the range of 1.3-2 MHz, and consists of pulses generated at a rate in the range 100-1000 Hz, with each pulse having a duration in the range 10-2000 microseconds. Aptly the power intensity of the ultrasound signal is no higher than 100 milliwatts per square centimetre. Aptly the ultrasound device is an EXOGEN (Trade Mark of Exogen, Inc.) ultrasound device.

The method of treatment should ideally be often, and ideally daily at least. It would be hoped that a treatment for 30 days would be sufficient. However the treatment may be for other time periods for example 10, 20, 25, 35 or 40 days. The duration of the treatment may vary and there is no set requirement. Typically the duration may be for approximately 10, 15 or 20 minutes each time.

Ideally the ultrasound would be directed to the cellular milieu where the infection occurs.

The ultrasound may be directed to particular cells of the body e.g. phagocytic cells but also bones and joints, bone marrow, the brain and the meninges and any other cell, tissue or part of the body.

In cases of infection like septicaemia the ultrasound signal could be directed to a prominent position on the body where a major blood artery was located in the surface of the body.

In cases of meningitis the ultrasound may be directed for example at the brain, the meninges, the cerebo-spinal fluid or any combination thereof. The ultrasound transducer could even be located inside the body to be treated to ensure that the maximum ultrasound signals reach the desired target e.g. cellular milieu.

Although the ultrasound signal could be directed at the cellular milieu ideally phogacytotic cells would be targeted by the ultrasound signal to enhance/increase their phagocytic activity and thus kill bacteria and heal the bacterial infection.

These phagocytotic cells may include all committed hematopanetic cells of the bone marrow lineage. Thus may include but not be limited to B-Lymphoid Progenitor cells, Pro-B and Prob-B-1 cells, Pre-B cells, Pre-B-II cells, Immature B cells, Mature B cells, CFU-Blast cells, GEMM cells, BFU-E cells, CFU-E cells, Pronormoblast cells, Reticulocyte cells, BFU-Mk cells, CRU-Mk cells, Megakargocyte cells, CFU-GM, CFU-G, Neutrophilic Myelocyte, Neutrophil cells, CFU-Mast cells, CFU-M cells, Dendritic cells, Mast cells, Monocyte Pre DCI cells, DCI cells, Macrophage.

The ultrasound may enhance phagocytosis of the mature or immature form of the above cells.

The ultrasound may stimulate professional and non-professional phagocytes.

According to the present invention there is provided a method for treating a bacterial and/or fungal infection comprising applying ultrasound to a bacterial infected site to increase the activity of phagocytic cells of the body.

According to the present invention there is provided a method for enhancing the activity of phagocytic cells.

According to the present invention there is provided a method for enhancing the activity of macrophage.

Further to the present invention there is provided a method for cytokine production and/or enhanced activity of cytokine comprising applying ultrasound to a cell, cells and/or tissue.

By stimulating a cell, cells and/or tissue with ultrasound there is an increase in cytokines. Increasing Cytokines, or increasing cytokine activity in a treated cell enhances activity of other cells in the environment to e.g. clear infection, kill bacteria and/or fungi.

Embodiments of the present invention also provides a method for MMP production and/or enhanced activity characterised by the step of applying ultrasound to a cell, cells and/or tissue.

MMP e.g. MMP2 and MMP9 breakdown the biofilm matrix of bacteria in a bacteria infection in or on for example a body, and therefore allow phagocytic cells to easily reach and destroy the bacteria. Increased MMP production or activity thus increases, enhances phagocytosis and thus helps clear bacterial infections/destroy bacteria in the body.

Further still the present invention provides a method for EMMPRIN production and/or enhanced activity of EMMPRIN characterised by the step of applying ultrasound to a cell, cells and/or tissue. Increased EMMPRIN or increased EMMPRIN activity gives increased production or activity of MMPs. The EMMPRIN gives increased production of activated MMPs.

The present invention may be used to treat any bacterial and/or fungal (Mycoses) infection, regardless of whether the infection is caused by:

Gram positive bacilli bacteria
Gram negative bacilli bacteria
Gram positive cocci bacteria
Gram negative cocci bacteria
Spirochetes
Yeast or any other type of bacteria or fungi that can be phagocytosed by phagocytic cells.

The present invention can be used to treat any bacterial or fungal infection including, but not limited to:
Actinomycosis—Anthrax—Aspergillosis—Bacteremia—Bartonella Infections—Botulism—Brucellosis—Burkholderia—Infections—Cellulitis—Campylobacter—Infections—Candidiasis—Cat Scratch Disease—Chlamydia Infections—Cholera—Clostridium—Infections—Coccidioidomycosis—Cross Infection—Cryptococcosis—Dermatomycoses—Diphtheria—Ehrlichiosis—Escherichia coli Infections—Fasciitis, Necrotizing—Fusobacterium—Infections—Gas Gangrene—Gram-Negative Bacterial Infections—Gram-Positive Bacterial Infections—Histoplasmosis—Impetigo—Klebsiella—Infections—Legionellosis—Leprosy—Leptospirosis—Listeria Infections—Lyme Disease—Maduromycosis—Melioidosis—Mycobacterium Infections—Mycoplasma Infections—Mycoses—Nocardia Infections—Onychomycosis—Ornithosis—Plague—Pneumococcal Infections—Pseudomonas Infections—Q Fever—Rat Bite Fever—Relapsing Fever—Rheumatic Fever—Ricksettsia Infections—Rocky Mountain Spotted Fever—Salmonella Infections—Scarlet Fever—Scrub Typhus—Sepsis—Sexually Transmitted Diseases, Bacterial—Staphylococcal Infections—Tetanus—Tuberculosis—Tularemia—Typhoid Fever—Typhus, Epidemic Louse-Borne—Vibrio Infections—Yaws—Yersinia Infections—Zoonoses—Zygomycosis—Osteomyelitis—Meningitis.

### BRIEF DESCRIPTION OF DRAWINGS

Embodiments of the invention will now be described by way of an example with reference to the following drawings.

FIG. 1 shows the effect of LIPUS on phagocytosis capacity in adherent human macrophages.

FIG. 2 shows the effect of LIPUS on cytokines release in adherent human macrophages.

FIG. 3 shows the effect of LIPUS on the synthesis and release of MMPs in adherent human macrophage.

FIG. 4 shows the effect of LIPUS on the expression of MMPs in adherent and suspended human macrophages.

FIG. 5 shows the effect of LIPUS on cell membrane-associated EMMPRIN in adherent human macrophages.

FIG. 6 shows the effect of LIPUS on cell membrane-associated EMMPRIN in adherent human macrophages.

FIG. 7 shows the effect of LIPUS on cell membrane-associated EMMPRIN in adherent and suspended human macrophages.

FIG. 8 shows the effect of LIPUS on the concentration of EMMPRIN in adherent human macrophages.

FIG. 9 shows the effect of LIPUS on tyrosine phosphorylation in adherent human macrophages.

FIG. 10 shows the effect of LIPUS on tyrosine phosphorylation in suspended human macrophages.

FIG. 11 shows the effect of LIPUS on Src, ERK and P38 MAPK phosphorylation in adherent human macrophages.

FIG. 12 shows the effect of LIPUS on the assembly of F-actin in adherent human macrophages.

FIG. 13 shows the effect of LIPUS on tyrosine phosphorylation in adherent human macrophages.

FIG. 14 shows the effect of LIPUS on phagocytosis of E. coli by J774A.1 mouse macrophages.

### DETAILED DESCRIPTION

### EXAMPLE 1

Cell Isolation and Culture

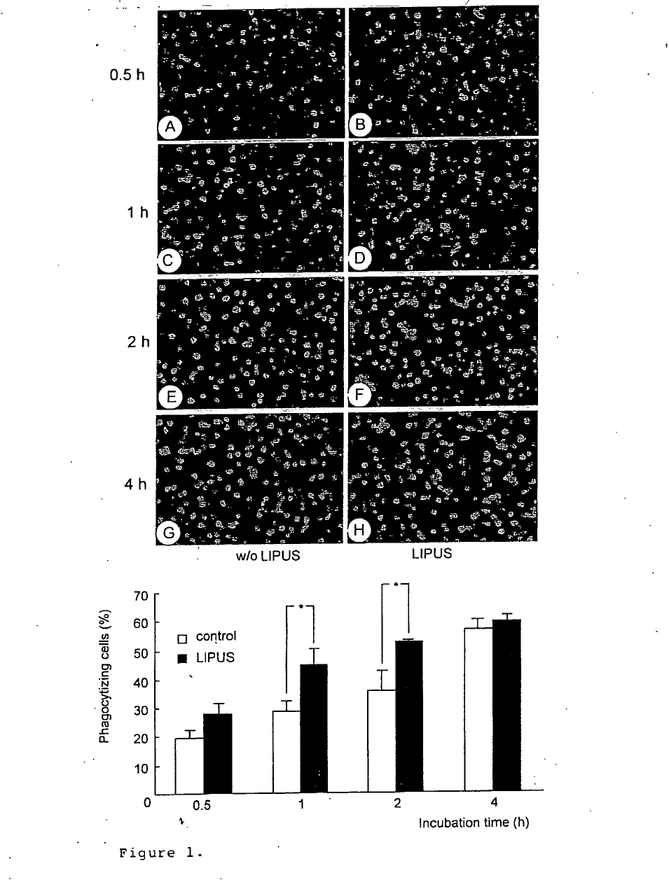

Mononuclear cells were isolated from buffy coats of healthy human blood donors by low-endotoxin Ficoll-Paque (d=1.077) density gradient centrifugation. Briefly, buffy coat was diluted 1:1 with calcium- and magnesium-free Dulbecco's phosphate-buffered saline (PBS). The diluted buffy coat was layered over the density gradient (Ficoll-Paque), and centrifuged at 800 g for 30 min at 23° C. Since erythrocytes and neutrophils are denser than Ficoll-Paque, they penetrate it and sediment to the bottom of the centrifuge tube. The lighter mononuclear cells (lymphocytes and Monocytes) sediment to the plasma—Ficoll-Paque interface. At equilibrium, the mononuclear cells were carefully aspirated from the interface, washed twice with PBS and once with RPMI-1640 medium (RPMI) to remove contaminated platelets, residual erythrocytes. The cells (1-2×10⁶ cells ml) were cultured in biofolie bags with X-VIVO 10 medium (X-VIVO) containing 10% fetal calf serum (FCS) and 2 mM _{L}-Glutamine under standard culture conditions (humidified 5% CO₂, 37° C.). After differentiation for 7-10 days in biofolie bags without any further manipulation, these cells were referred to as original macrophages from here on. The procedure for cell isolation and culture is also summarized in FIG. 1. Isolation and culture of human blood macrophages. Buffy coat (A) was diluted in PBS and centrifuged with Ficoll-Paque, then the mononuclear layer (marked with yellow ellipse, B) was seeded in biofolie bags with X-VIVO containing 10% FCS(C) and cultured for 7-10 days. Thereafter, the original macrophages were seeded in six- well plates (D). The morphology of adherent macrophages is shown as the picture E.

Adherent Macrophages

Original macrophages were mechanically scaped to detach the cells, and seeded (2×10⁶ cells/well) in six-well plates and glass bottom microwell dishes (35 mm). For immunifluorescence microscopy, cells were seeded on 1 cm² glass coverslips in six-well plates. All the plates and dishes were coated with fibronectin (2 μg/ml) overnight at 4° C. prior to use. Cells were cultured with 1 ml RPMI containing 10% FCS, 2 mM _{L}-Glutamine, 100 IU/ml penicillin, and 100 μg/ml streptomycin at 37° C. in a 5% humidified incubator. From now on cells were allowed to attach for 2 h, and carefully washed with RPMI twice to remove non-adherent cells. These remaining cells were called adherent macrophages.

Suspended Macrophages

Original macrophages were detached as described above and transferred into centrifuge tubes. Cells were washed twice with RPMI to remove FCS and seeded (2×10⁶ cells/well) in six-well plates with 2 ml RPMI containing 2 mM _{L}Glutamine, 100 IU/ml penicillin, and 100 μg/ml streptomycin, but without FCS. The cells were taken as suspended macrophages. To keep cells in suspension, fibronectin (2 μg/ml) was added immediately after cells seeded, and cells were stimulated within 2 min after seeding. Stimulation with low intensity pulsed ultrasound (LIPUS). In the present study, a modified SAFHS (Trade Mark of Exogen, Inc.) system was used, which produces a 1.5 MHz ultrasound wave, 200 μs pulse modulated at 1 kHz, with an output intensity of 30 mW/cm². The equipment comprises two parts: (i) main operating unit, and (ii) transducers mounted on a cell culture plate. The main operating unit drives six transducers simultaneously when all tranducers were connected. All six transducers are mounted in an assembly that matches the well positions of a six-well plate. The correct function of the equipment was checked before each experiment using an ultrasound-activated LED indicator supplied by the manufacturer. To prevent chilling the cultures the ultrasound transducers and the coupling gel were warmed in the CO₂-incubator prior to stimulation.

The six-well plate with macrophages was placed on ultrasound transducers using the coupling gel. Air bubbles between the plate and the transducers were forbidden. The untreated plates were always put in a separate incubator.

The low intensity pulsed ultrasound equipment used was a Sonic Accelerated Fracture Healing System (SAFHS Trade Mark of Exogen, Inc.). It consists of A: main operating unit; B: ultrasound transducers; C: ultrasound activated LED indicator; D: coupling gel; E: six-well plate with medium.

Detection of Macrophages Capacity to Phagocytose FITC-Labelled E. coli

Macrophages were seeded in glass bottom microwell dishes (2×10⁶ cells/dish) pre-coated with fibronectin. Cells were incubated with FITC-E. coli (1.5×10⁷ bacteria/dish) at 37° C. At various time points the fluorescence of noningested bacterial were quenched by trypan blue (0.25%, diluted in PBS), and the digital images were taken by fluorescence microscopy. To quantify phagocytosis capacity, at least 200 cells of each image were counted, and three images per condition were assessed. The percentage of phagocytozing cells represents the ratio of the number of cells containing internalised bacteria to that of the total cells in the same image.

Cytokine Proteins Array

Cytokine array membranes precoated with 79 cytokine antibodies by the manufacturer were separately blocked with 2 ml 1× blocking buffer for 30 min at room temperature (RT) in eight-well tray, then incubated with 1 ml serum-free conditioned media with gentle agitation overnight at 4° C. Membranes were washed for 5 min three times with wash buffer I and twice with wash buffer 11, subsequently incubated with 1 ml of biotin-conjugated antibodies (1:250, diluted in blocking buffer) for 3 h at RT. After washing extensively with wash buffer, the membranes were incubated with HRP-conjugated streptavidin (1:1000, diluted in blocking buffer) for 40 min at RT. The signals were visualized and exposed to Kodak imaging films. The blots were scanned using the BioRad Gel Doc 1000 instrument and the intensities of the signals were expressed in relation to the corresponding controls. The data are shown as the fold increase above control.

Fluorometric DNA Measurement

Cells were washed with PBS and lysed by freeze-thaw cycles (3×). Thereafter cells were incubated with 0.25% trypsin solution (containing 0.01% ethyenediaminetetraacetic acid, EDTA) (1 ml/well) for 30 min at 37° C. To reduce the viscosity cells were sonicated for 10 sec twice. Standards (Calf thymus DNA stocking solution diluted in DNA buffer, the concentrations are 0, 0.05, 0.125, 0.25, 0.5, 1 and 2 μg/ml) and samples were transferred to 24-well plates and the volume was adjusted to 400 μl with DNA buffer. Hoechst 33258 (2 μg/ml, 1.6 ml/well) was added to each well followed by incubation with gentle agitation for 20 min in the dark at RT. Time-resolved fluorescence was measured using a Victor 1420 Multilabel Counter (excitation) wavelength: 360 nm, emission wavelength: 460 nm). Sample concentrations were calculated according to the standard curve. All measurements (standards and samples) were obtained in duplicate. Variations of duplicate measurements were usually between 0.5% and 5% and did not exceed 8%.

Immunofluorescence Microscopy

Detection of Membrane-Associated EMMPRIN

Cells were cultured on glass coverslips for 3 days and starved with 0.1% FCS overnight followed by stimulation with LIPUS. 48 h later, the cells were washed twice with PBS and once with H₂O rapidly, and fixed with acetone for 20 min at RT. After the coverslips were mounted on slide with silicone, cells were treated by 0.3% H₂O₂ (diluted in methanol) for 30 min to block endogenous hydrogen peroxidase, then non-specific binding sites were blocked by TNB buffer for 30 min. The following steps were performed sequentially - primary antibody (mouse anti-human- EMMPRIN, Serotec, 1:1000, 15 min) secondary antibody (HRP-anti-mouse, 1:1000, 45 min), biotin-TSA-reagent (diluted 1:1500 in Diluent, 15 min), streptavidin-FITC (1:1000, 30 min), and Hoechst 33258 (2 mg/ml, 15 min). All incubations were performed in a humid chamber at RT. TNB buffer was used for dilution except additional mention. Slides were thoroughly washed after each step with 0.05% Tween 20 in PBS (PBST) three times each for 5 min. The photos were taken by the fluorescence microscopy. To compare different staining intensities, exposure time was always the same. Non-specific staining was controlled by using mouse IgG as a substitute for specific primary antibody.

Detection of F-Actin and Tyrosine Phophorylated Proteins

Cells were cultured on glass coverslips for 3 days and starved with 0.1% FCS overnight. After treatment with 10 min LIPUS, cells were stopped 40 min. The cells were washed twice in PBS and fixed with 4% paraformaldehyde for 20 min at RT. After the coverslips were mounted on slide with silicone, cells were treated by 0.3% H₂O₂ (diluted in PBS) for 30 min block endogenous hydrogen peroxidase, then permeabilized with 0.2% Triton X-100 for 10 min. Non-specific binding was further blocked with FCS in PBS (1:1) for 30 min. To detect tyrosine phosphorylated proteins, the following steps were performed sequentially: primary antibody (mouse anti-human-phosphotyrosine, diluted 1:100, 1 h), secondary antibody (HRP-anti-mouse, 1:100, 1 h) biotin-TSA-reagent (1:500 in Diluent, 15 min), streptavidin-FITC (1:100, 30 min), and Hoechst 33258 (2 mg/ml, 15 min). To detect F-actin, glass coverslips were incubated with Alexaphalloidin (1:400) for 1 h and nuclei were stained with Hoechst 33258 (2 mg/ml, 15 min).

Gelatin Zymography

To perform zymography cells were cultured in the absence of FCS and supernatants were collected 24 h or 24 h after LIPUS treatment, centrifuged at 1000 rpm for 5 min to eliminate insoluble pellets and stored at −20° C. until assayed.

To prepare gels containing 0.2% gelatine, 20 mg/ml gelatine was heated for 90° C. for 1 hr, then clarified by centrifugation at 4000 rpm for 5 min at RT, thereafter the gelatine solution (1 ml) was added to 7.5% SDS-PAGE gel mix (9 ml) before polymerisation. The conditioned medium was diluted in zymogram sample buffer and mixed well on vortex to prepare loading samples. Thereafter electrophoresis was performed in tris-glycine buffer for 3-4 h at 90 V on ice, subsequently the gels were soaked in 100 ml zymogram renaturing buffer for 15 min twice at RT to remove SDS. Then the gels were incubated in 200 ml zymogram developing buffer at 37° C. overnight. The proteolytic activity was shown by staining with 0.34% Coomassie blue R-250 for 30-60 min, and destaining with 15% (v/v) acetic acid and 40% (v/v) methanol. Areas of protease activity appeared as clear bands against a dark blue background. Proteolytic bands were scanned using the RioRad Gel Doc 1000 System and the bands were quantified by densitometry.

Quantitation of Soluble EMMPRIN

Sample preparation was the same as that for gelatine zymography. Soluble EMMPRIN was measured by time-resolved fluorescence immunoassay (TR-FIA). Briefly, 96-well microtiter plates were coated with rabbit-anti-mouse IgG (50 μl/well, 6 μg/ml diluted in coating buffer) at 4° C. overnight. Then cells were washed three times with TNT buffer followed by incubation for another 4 h with capture antibody (mouse-anti-human EMMPRIN, R & D, 0.25 μg/ml diluted in coating buffer) and blocked with assay buffer (250 μl/well) for 2 h at RT. Standards (serially diluted in assay buffer, the concentrations are 40, 20, 10, 5, 1.5, 1.25, 0.625, 0.3125, 0 ng/ml) and samples were loaded and incubated at 4° C. overnight. After washing thoroughly, the plates were incubated for 4 h with detection antibody (biotinylated h EMMPRIN affinity purified goat IgF, 0.05 μg.ml diluted in assay buffer). The next step was the incubation with europium-labelled streptavidin (diluted 1:500 in assay buffer) for 1 h followed by another incubation with enhancement solution for 45 min at RT. Thereafter time-resolved fluorescence was measured using Victor 1420 Multilable Counter (excitation wavelength: 340 nm, emission wavelength: 615 nm). Sample concentrations were calculated using spline function of standard curve. The volumes of reagents and samples used were 100 μl/well unless otherwise stated. All measurements (standards and samples) were obtained in duplicate. Variations of duplicate measurements were usually between 0.5% and 5%, and did not exceed 8%.

Detection of Cell Membrane Associated EMMPRIN

Macrophages were washed twice in ice-col PBS and incubated with EZ-Link™ Sulfo-NHC-LC-LC-Biotin (Biotin) (10 μl in 1 ml PBS per well) for 30 min on ice with gentle agitation, then lysed with radioimmune precipitation assay (RIPA) buffer (250 μl/well) for another 30 min on ice. Cells were scraped off and transferred into microcentrifuge tubes followed by centrifugation at 13,000 rpm for 10 min at 4° C. to remove insoluble pellets. The samples were called biotinylated samples and immunoprecipitated by EMMPRIN mAb prior to electrophroresis. The samples directly lysed with RIPA buffer without biotin treatment were called unbiotinylated samples. The unbiotinylated samples could be used for electrophoresis without immunoprecipitation.

The biotinylated samples were first cleared by incubation with 60 μl of packed protein-A conjugated sepharose with agitation for 2 h at 4° C. Then supernatants were incubated with 2.5 μg of EMMPRIN monoclonal antibody (mAb) overnight with agitation at 4° C. and incubated with protein-A conjugated sepharose for another 1.5 h to precipitate immune complexes. The beads were then washed 7 times with Wash buffer A (×2), Wash buffer BI (×1), Wash buffer BII (×1), Wash buffer C (×2), and ddH₂O (×1), respectively. Immune complexes eluted from beads or the unbiotinylated samples were mixed with 2× reducing Laemmli sample buffer and boiled at 95° C. for 5 min. Supernatants from biotinylated mixes or unbiotinylated mixes were subjected to 10% SDS-PAGE under constant 21 mA for 2 hr. Protein marker was used to indicate the protein molecule size. After elctrophoresis proteins were transferred to PVDF member with semi-dry electrotransfer system under constant 0.8 V/cm² of PVDF membranes for 2 h. Non-specific binding was blocked by the incubation of the membranes in 5% albumin solution overnight at 4° C. For biotinylated samples, next was the incubation with streptavidin-HRP (1:2000) for 45 min at RT, while for the unbiotinylated samples, next was the incubation with EMMPRIN mAb (1:5000, R & D) for 2 h and anti-mouse-HRP (1:2000) for 1 h at RT. Finally EMMPRIN and protein marker were visualized by the chemiluminescence detection system and exposed to Kodak imaging films.

Detection of Tyrosine Phosphorylated Proteins, Src, ERK, and p38 MAPK

To avoid any interference from movement, the plate with serum-starved cells was put on the ultrasound transducers for 2.5 h to make culture thoroughly quiescent prior to stimulation. At indicated time after LIPUS stimulation cells were washed in ice-cold PBS and incubated with Lysis buffer for 5 min on ice. Then the cells were scraped off the plate and transferred in microcentrifuge tubes on ice. To shear DNA and reduce sample viscosity, the extract was sonicated for 10 sec three times in ice-cold water. After removing the insoluble pellets by centrifugation with 13,000 rpm for 10 min at 4° C., the lysates were mixed with 2× Laemmli sample buffer and boiled at 95° C. for 5 min. The proteins were separated by SDS-PAGE gel (8% for tyrosine phosphorylated proteins, 10% for Src and MAPKs). Electrophoresis was performed for 10 h under constant 60 V. Then proteins were transferred to PVDF membranes as described previously. After blocked with 5% albumin in 0.1% PBST, the blots were incubated with the primary antibodies—phosphor-tryrosine (4G10) (1:1000), phosphor-Src (Tyr416) (1:1000), phosphor-p42/44 MAPK (Thr202/Tyr204) (1:2000), or phosphor-p38 MAPK (Thr180/Tyr182) (1:2000) at 4° C. overnight, respectively. After further incubation with corresponding HRP-conjugated secondary antibody (1:2000) for 1 hr, the bands were visualized using enhanced chemiluminescence Western blotting system according to the manufacturer's instructions. All the antibodies were diluted in blocking buffer.

To detect the total knase proteins as loading control, after being probed for phosphorylated proteins the blots were incubated in stripping buffer at 45° C. for 1 h and washed in 0.1% PBST three times each for 5 min. Then the blots were blocked in 5% Albumin for 30 min at RT and reprobed with Src (1:1000), p42 MAPK (1:2000) and p38 MAPK mAb (1:2000) for total Src, p42 MAPK, and p38 MAPK, respectively. The next procedure was the same as the detection of phosphorylated protein.

Statistical Analysis

At least three independent experiments were performed in triplicate for each result, using cells from different donors. Values in bar diagrams were expressed as the mean of the triplicates. Data were presented as mean±standard deviation (SD). Statistical significance was evaluated using one-way-ANOVA (Scheffe-test) for comparison between the control and test groups. Values were considered to be statistically different when P<0.05.

Results

Effect of LIPUS on Macrophages Phagocytosis Capacity to Phagocytose E. coli

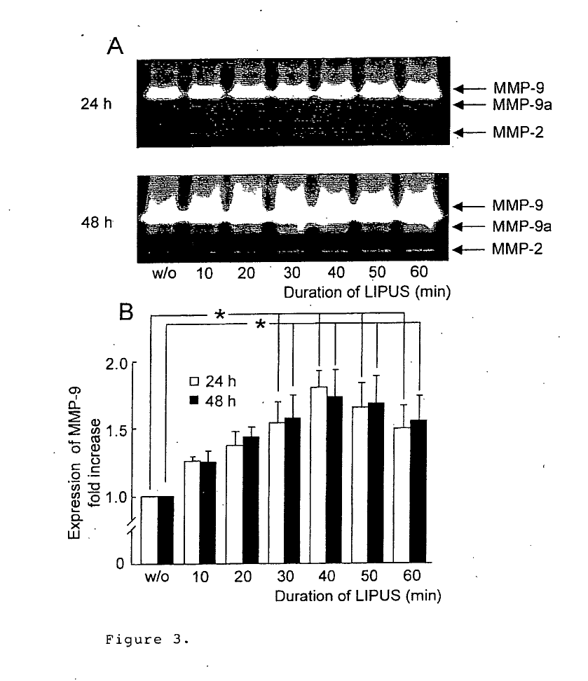

To study the effect of LIPUS on macrophage phagocytosis, we incubated cells grown on glass bottom microwell dishes with FITC-E. coli for 0.5, 1, 2, and 5 h, and quenched by trypan blue. The images were taken to determine the numbers or phagocytozing cells (green) in relation to that of corresponding total cells (red and green). The percentage of phagocytozing cells is defined as phagocytosis capacity. As shown in FIG. 1, phagocytozing cells were increased in a time-dependent manner whether cells were exposed to LIPUS or not. Phagocytosis capacity was significantly increased by LIPUS at 1 and 2 h compared to untreated cells (44.77±5.44 s 28.71±3.71; 52.39±0.89 vs 35.47±7.41, n=9, FIG. 3B).

Effect of LIPUS on Cytokine Synthesis

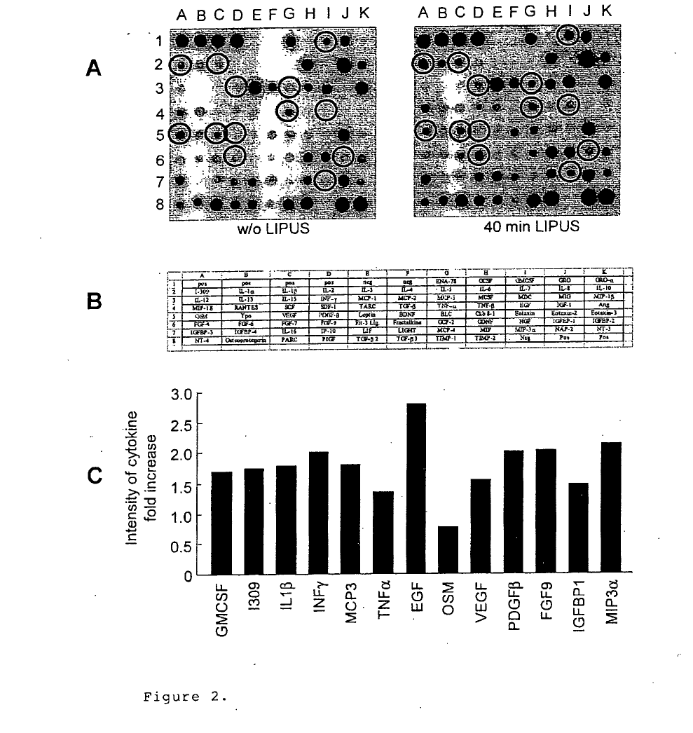

To demonstrate the effect of LIPUS on the synthesis and release of soluble cytokines, array membranes were incubated with conditioned media of control and stimulated macrophages. The signals of 79 cytokines were detected with biotinylated antibodies. Serum starved human macrophages were stimulated with 40 min LIPUS and the culture supernatants were collected 24 h after stimulation. The samples were incubated with arrayed antibody supports followed by incubation with biotinylated antibodies and HRP-conjugated streptavidin. Then the membranes were incubated with detection buffer and exposed to the films. A: Images are the representative of three independent experiments. The signals marked were presented as the ratio of density in C. B: Human cytokine array map. C: Quantitation of the signals was performed by densitometry. The data are shown as the ratio of the density (LIPUS/control). As shown in FIG. 2, LIPUS increased some cytokines (e.g. GM-CSF, I-309, IL-1 β, INF-y, MCP-3, TNF-a, EGF, VEGF, PDGF-β, FGF-9, IGFBP-1, and MIP-3a) expression 24 h after stimulation, simultaneously; LIPUS decreased the expression of Osm and Tpo.

With regard to the results of FIG. 1 macrophages were seeded in glass bottom microwell dishes and incubated with FITC-E. Coli for 0.5, 1, 2, and 4 h, respectively. Thereafter fluorescence of non-ingested bacteria was quenched by trypan blue. The images were taken by the fluorescence microscopy and cells were counted. At least 200 cells of each image were counted, and three images per condition were assessed. Numerical data were presented as mean±SD in three independent experiments (n=9). *P<0.05 versus corresponding control.

Influence of LIPUS Duration

To investigate the effect of LIPUS durations on the expression of MMPs, macrophages were exposed to LIPUS for 10, 20, 30, 40, 50, and 60 min, respectively. Conditioned media were collected 24 h and 48 h after stimulation.

With regard to the effect of LIPUS on the synthesis and release of MMPs in adherent human macrophages, serum starved human macrophages were stimulated with LIPUS for 10, 20, 30, 40, 50, and 60 min. Conditioned media were collected after 24 h and 48 h and analysed by gelatin zymography (A). Quantitation of the data was performed via the densitometry (B). The data shown in FIG. 3 are the fold increase above control in three independent experiments and are mean±SD. *P<0.05 compared to control cells. As shown in FIG. 3, constitutive expression of MMP-9 was much higher than that of MMP-2, and the expression of MMP-9 was markedly increased when incubation time was prolonged (48 h versus 24 h). For adherent macrophages, LIPUS augmented the expression of pro-MMP-9 and its active form. Treatment with LIPUS for 10, 20, 30, and 40 min increased the expression of MMP-9 gradually, showing a peak at 40 min, and thereafter a decline at 50 and 60 min. In our system, LIPUS also enhanced the release of MMP-2.

A Comparison between Adherent Macrophages and Suspended Macrophages

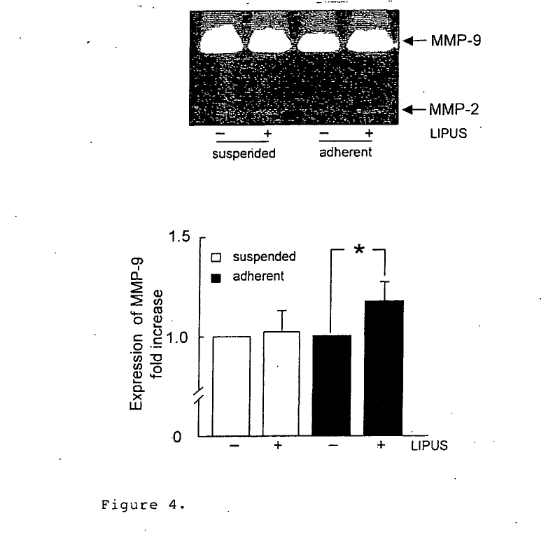

To examine whether the cell adhesion is necessary to induce MMPs by LIPUS, adherent and suspended cells were stimulated with 40 min LIPUS. The conditioned media were collected 48 h after stimulation to demonstrate the expression of MMPs using gelatin zymography. As shown in FIG. 4, LIPUS increased the expression of MMP-9 in macrophages.

Greater increases were observed in adherent macrophages compared to suspended macrophages.

With regard to the results shown in FIG. 4 human macrophages were seeded on the six-well plates and stimulated with LIPUS for 40 min immediately (suspended cells) or 2 h later (adherent cells) in the absence of FCS. Conditioned media were collected after 48 h and analysed by gelatin zymography (upper panel). The data are quantified by densitometry and are shown the fold increase above control (mean±SD) in three independent experiments (lower panel). *P<0.05 compared to control cells.

Effect of LIPUS on EMMPRIN Protein Expression

To investigate the effect of LIPUS on EMMPRIN, we detected cell membrane-associated and soluble EMMPRIN.

Influence of LIPUS Duration

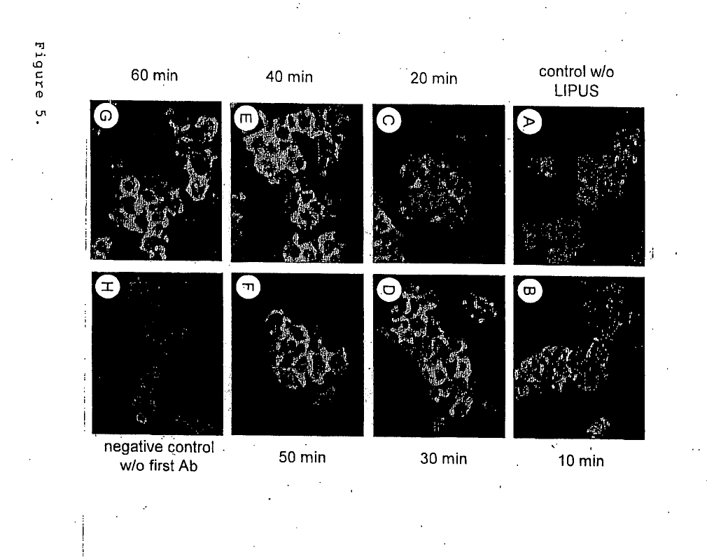

Intensive staining patterns of EMMPRIN were observed in cultures stimulated with LIPUS for indicated times. EMMPRIN (green) was distributed on the cell membrane. The images (FIG. 5) showed that LIPUS (40, 50, and 60 min) strongly increased the EMMPRIN expression on the cell surface, which was further confirmed by Western blotting.

With regard to FIG. 5 serum starved human macrophages grown on glass coverslips were stimulated with LIPUS for indicated times. The cultures were stopped 48 h after stimulation, and fixed with acetone. EMMPRIN was stained by EMMPRIN mAb and Streptavidin-FITC (green), nuclei stained with Hoechst 33258 (blue). A, B, C, D, E, F and G: 0, 10, 20, 30, 40, 50 and 60 min (duration of LIPUS stimulation), H: negative control (EMMPRIN mAb was replaced by Mouse IgG).

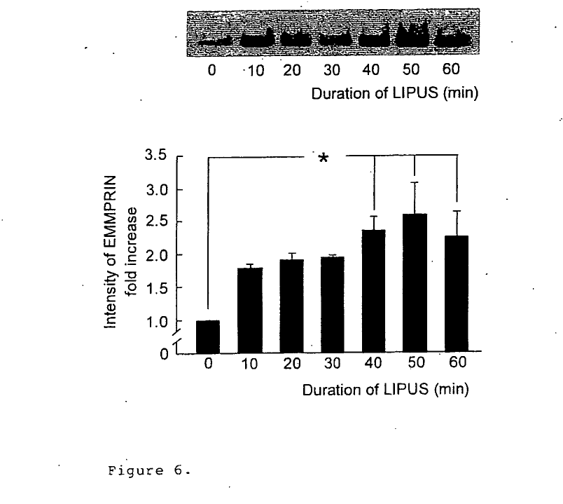

As shown in FIG. 6, cell membrane-associated EMMPRIN was increased gradually from 10 to 50 min LIPUS duration, and decreased at 60 min.

With regard to the results of FIG. 6 serum starved human macrophages were stimulated with LIPUS for indicated times. 48 h later, cell membrane proteins were biotinylated using the membraneimpermeable sulfo-NHS-LC-LC-Siotin and lysed with RIPA buffer. Cell membrane-associated EMMPRIN was immunoprecipitated using EMMPRIN mAb and protein A-sepharose. Immunoprecipitates were resolved by 10% SDS-PAGE and biotinylated proteins were detected by HRP-conjugated-streptavidin. The quantitative data were obtained by densitometry, and shown as the fold increase above control (mean±SD) in three independent experiments. *P<0.05 compared to control cells. It is interesting that the tendency is just the opposite to that of soluble EMMPRIN, but similar to that of MMP-9 (FIG. 3).

A Comparison between Adherent Macrophages and Suspended Macrophages

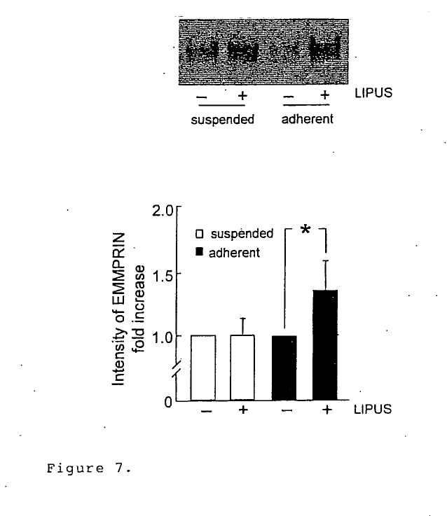

The expression of EMMPRIN was examined in the cell lysate of adherent and suspended cells by Western blotting. As shown in FIG. 7, 40 min LIPUS induced an increase of EMMPRIN in adherent cells, to a greater effect than was observed in suspended cells (FIG. 7). With regard to the results of FIG. 7 human macrophages were seeded in the six-well plates and stimulated with 40 min LIPUS, immediately (suspended cells) or 2 h later (adherent cells). After 48 h cells were lysed with RIPA buffer, the lysates were resolved by SDS-PAGE on 10% gel. EMMPRIN in the total cell lysates was detected by EMMPRIN mAb. By densitometry the data shown are the fold increase above control in three independent experiments, and are mean±SD. *P<0.05 compared to control cells.

Effect of LIPUS on Soluble EMMPRIN

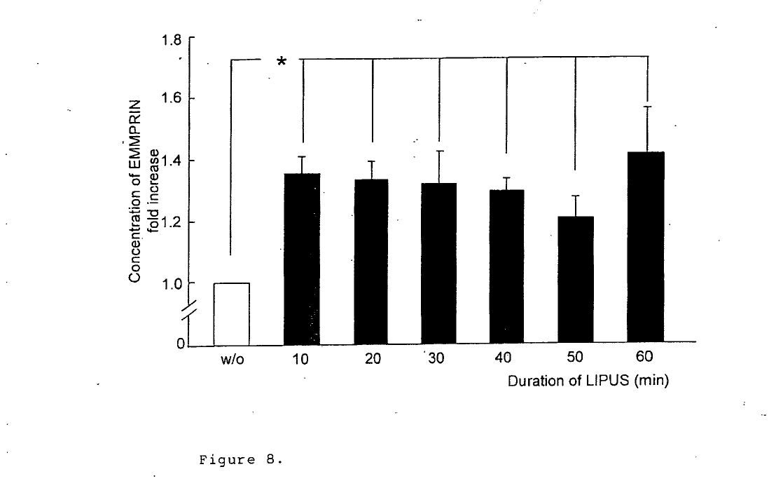

To quantify soluble EMMPRIN, supernatants were collected 48 h after stimulation, and the concentration of EMMPRIN was measured by time-resolved fluorescence immunoassay (TR-FIA), finally the value of EMMPRIN was corrected by corresponding DNA concentration. As shown in FIG. 8, LIPUS enhanced the concentration of soluble EMMPRIN.

With regard to the results in FIG. 8 serum starved human macrophages were stimulated for the indicated times by LIPUS. 48 h later, supernatants were collected and soluble EMMPRIN was measured using TR-FIA. The data shown are the fold increase above control in three independent experiments, and are mean±SD. *P<0.05 compared to control cells.

Effect of LIPUS on Tyrosine Phosphorylation in Adherent Macrophages

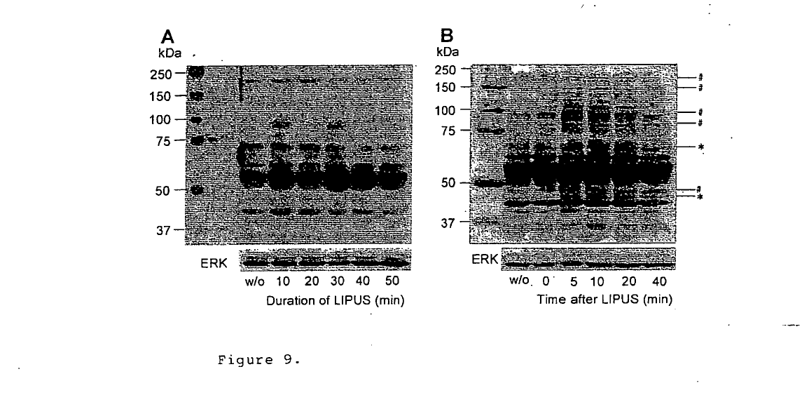

To find the LIPUS duration showing the strongest effect on tyrosine phosphorylation, serum starved adherent macrophages were stimulated by LIPUS for 10, 20, 30, 40, and 50 min and stopped directly after stimulation. As shown in FIG. 9A, tyrosine phosphorylation of several proteins was increased using 10 and 30 min LIPUS. Therefore, in the following experiments we used 10 min LIPUS. Adherent macrophages were challenged with 10 min LIPUS and stopped at 0, 5, 10, 20, and 40 min after stimulation to detect the tyrosine phosphorylated proteins. Maximum phosphorylation of most proteins was seen in the first 10 min after LIPUS (marked with #, FIG. 98). However, some proteins showed their maximum phosphorylation 40 min after LIPUS (marked with *, FIG. 9B). With regard to the results shown in FIG. 9 human macrophages were stimulated by LIPUS for indicated time after starvation and cultures were stopped at required times after stimulation. Cells were lysed, and immunoblotted with phospho-tyrosine mAb (4G10). Thereafter the stripped blots were reprobed with p42 MAPK mAb (protein loading control). The positions of molecular weight markers are indicated on the left side, 9A: Cells were stimulated by various LIPUS durations and stopped directly: 98: Cells were stimulated by 10 min LIPUS and stopped at indicated times after stimulation. Effect of LIPUS on tyrosine phosphorylation in suspended macrophages.

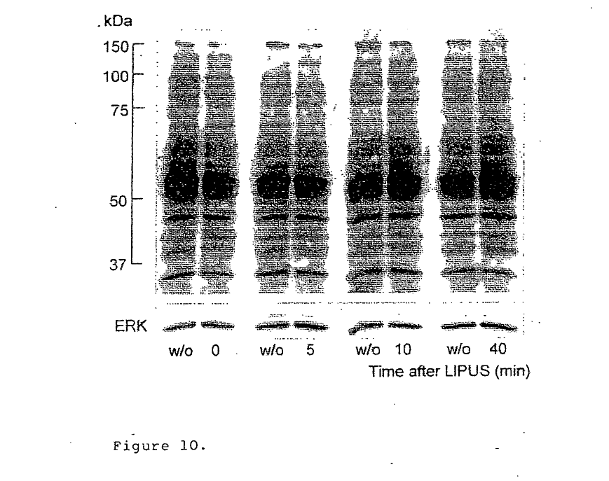

To demonstrate that cell-ECM contact is a prerequisite for LIPUS to induce cellular reactions, macrophages in suspension were stimulated by LIPUS for 10 min and tyrosine phosphorylation was studied. To obtain suspended macrophages, cells were immediately treated by LIPUS after cells were detached from the biofolie bags and seeded in the plates. In adherent macrophages a striking increase in tyrosine phosphorylation of multiple proteins was observed (FIG. 98). However, in suspended macrophages, no difference was detected between LIPUS treated cells and corresponding untreated cells (FIG. 10).

With regard to the results of FIG. 10 human macrophages were seeded in the six-well plates and stimulated immediately with 10 min LIPUS. Cultures were stopped at indicated times. Cells were lysed, and immunoblotted with phospho-tyrosine mAb (4G1 0). Then the stripped blots were reprobed with p42 MAPK mAb (protein loading control). The positions of molecular weight markers are indicated on the left side.

Effect of LIPUS on Phosphorylation of Src, ERK and p38 MAPK

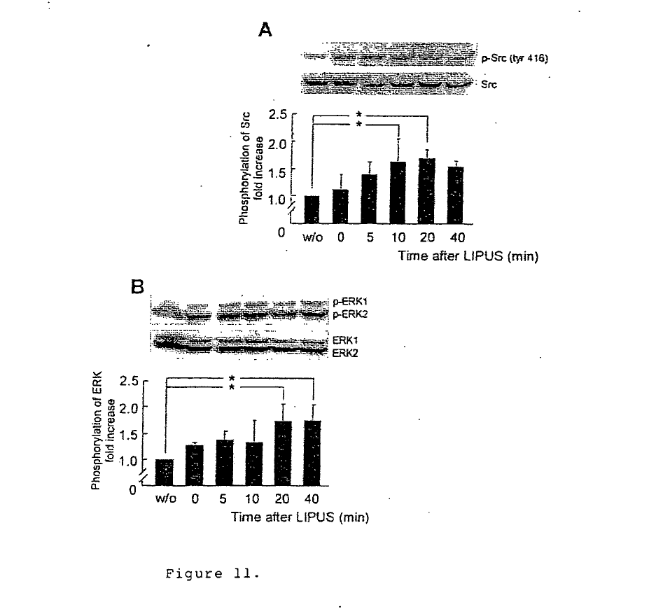

The phosphorylation of Src, ERK and p38 MAPK were analysed by Western blotting using specific monoclonal antibodies against phophorylated Src, ERK, and p 38 MAPK, respectively. Thereafter the blots were stripped and reprobed with corresponding antibodies against Src, ERK, or p38 MAPK to detect total Src, ERK, or p38 MAPK as protein loading control. Src phosphorylation was slightly increased directly at the end of LIPUS stimulation, and peaked between 10-20 min after stimulation (FIG. 11 A). In addition, LIPUS caused significant threonine and tyrosine dual phosphorylation of ERK at 20 min to 40 min post LIPUS stimulation, which was later than Src activation (FIG. 11 B).

With regard to the results shown in FIG. 11 serum starved human macrophages were stimulated with 10 min LIPUS and stopped at indicated times. Total cell lysates were prepared and subjected to 10% SDS-PAGE. The blots were probed with phospho-Src mAb (Tyr416) (11A), phosphop42/44 MAPK (ERK½) mAb (11 B). The stripped blots were reprobed with anti-Src, anti-p42 MAPK, and antip38 MAPK (protein loading control). Quantitation of phosphorylation was performed by densitometry. The data are shown as the fold increase above control (mean±SD) in three independent experiments. *P<0.05 compared to control cells.

Effect of LIPUS on the Formation of Focal Complexes

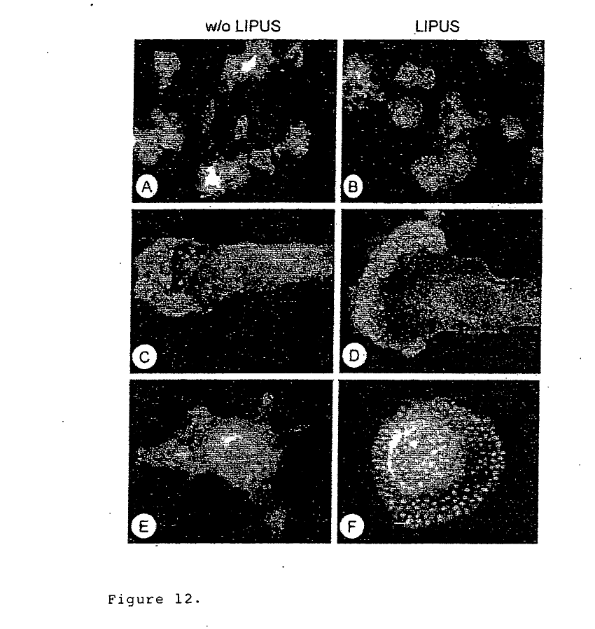

To demonstrate the effect of LIPUS on the formation of focal complexes, fluorescence staining of F-actin using Alexa-phalloidin and tyrosine-phosphorylated proteins using the phospho-tyrosine mAb (4G10) was performed. F-actin and tyrosine-phosphorylated proteins were presented as punctate structures, but few actin cables could be detected in macrophages. After starvation F-actin appeared at the periphery of the cells and distributed diffusely in the cytoplasm (FIG. 12 A, C, and E).

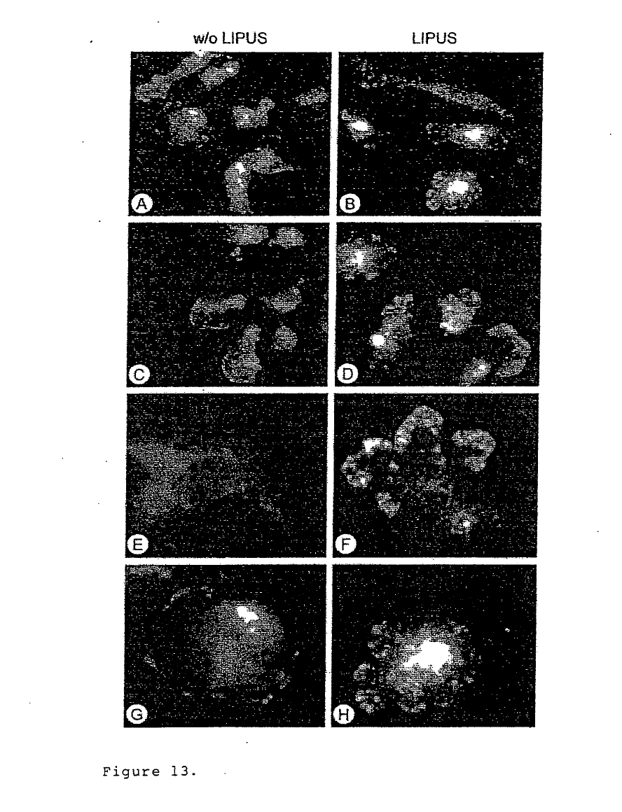

With regard to the results shown in FIG. 12 starved human macrophages grown on glass coverslips were stimulated with 10 min LIPUS. The cultures were stopped 40 min after stimulation, and fixed with 4% paraformaldehyde. F-actin was stained by Alexaphalloidin 12A, 12C and 12E: without LIPUS stimulation, 12B, 12D, and 12F: 40 min after LIPUS stimulation, 12A, 12B: ×100; 12C, 12D, 12E, 12F: ×600. Forty minutes after LIPUS stimulation, F-actin polymerisation was markedly induced and presented as an increase in the number and intensity of punctate structures (FIG. 12 B, D, and F). Tyrosine phosphorylated proteins were seen on the adhesion sites (FIG. 13). In control macrophages, few tyrosine-phosphorylated protein were localized at the periphery of the cells (FIG. 13A, C, E, and G). But when the cells were stimulated with LIPUS, more tyrosine-phosphorylated proteins appeared at the cell-substrate contacts (cell surface), (FIG. 13, B, D, F, and H). Taken together, LIPUS induced the formation of focal complexes. Ultrasound appears to organise cytoskeletal proteins, which in turn makes the cytoskeletal proteins more active. The cytoskeletal proteins are more active in an organised state.

With regard to the results shown in FIG. 13 starved human macrophages grown on glass coverslips were stimulated with 10 min LIPUS. The cultures were stopped 40 min after stimulation, and fixed with 4% paraformaldehyde. Tyrosine phosphorylated proteins were stained by phospho-tyrosine mAb (4G10). A, C, E and G: without LIPUS stimulation, B, D, F and H: 40 min after LIPUS stimulation. A, B, C, D: ×100; E, F, G, H: ×600.

### EXAMPLE 2

Phagocytosis

I. Primary Macrophage Purified and Differentiated from Peripheral Blood Monocytes.

II. Macrophage Cell Line

J774A.1 mouse macrophages were cultured in RPMI 1640 supplemented with 10% FCS in 24 well plates (1×10⁵/well) for 24 h. E. coli (K-12 strain)-FITC (Molecular Probes, Eugene, Oreg.) opsonized with human serum were added to the culture (E. coli:cells 15:1) directly before US stimulation. Macrophages were stimulated with US for 20 min (in 24-wells). Phagocytosis was stopped at 15, 30, or 60 min after US by washing the cells with PBS twice. Cell surface bacteria were quenched by 0.2% Trypan blue (Sigma) for 5 min, followed by fixation with 3.7% formaldehyde for 10 min. Thereafter, the nuclei were stained with 2 μg/ml Hoechst 33258 (Sigma) for 15 min. 5-8 photos were taken from each well. The average FITC and Hoechst 33258 density of each photo was measured with the Cell® Image Analysis. Phagocytosis was defined as the ratio of FITC to Hoechst 33258.

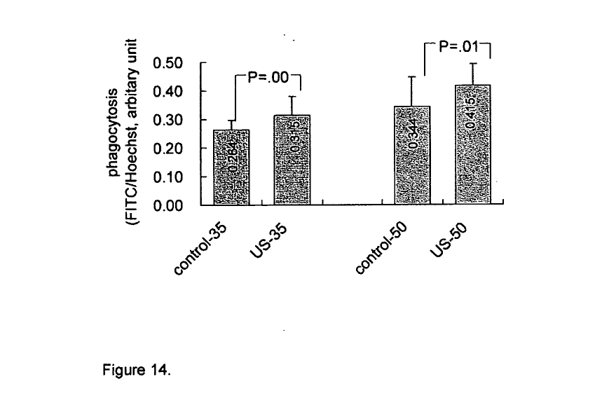

As shown in FIG. 14, ultrasound stimulation for 20 min was sufficient to increase phagocytosis by J774A.1 macrophages about 20% at 15 min to 30 min after US compared to control.

Discussion

Optimization of the procedure to isolate and culture macrophages.

Macrophage is a powerful research model in the fields of haematology, immunology, and other biology fields. The common procedure is that, monocytes are isolated by density gradient centrifugation, purified by immune selection of specific surface proteins, adherence, and cell size. Thereafter, purified monocytes are differentiated into mature macrophages variable from donor to donor and from researcher to researcher. For example, low yield, other leukocytes contamination, and loss of specific sub-population of macrophages frequently occur. An ideal method possessing the advantage of simplicity, purity, and high yield, does not exist. We propose here a relatively stable method to isolate mononuclear cells and culture macrophages. In the present culture system, after gradient centrifugation mononuclear cells were cultured for 7-10 days in the presence of autologous lymphocytes and platelets in biofile bags before being plated quantitatively, then purified by adherence to fibronectin.

Our culture system has the following characteristics. First, we incubated the complete mononuclear cell fraction biofile bags without any further purification, which weaken the shortcoming of low yield and variable adherence rate in many conventional techniques. Because cells grown in biofile bags are easily transferred to the plates, trypsination is not necessary for next plating, which also simplify the process to avoid cell loss. Second, our system incorporates the fact that lymphocytes and platelets may play pivotal roles in all stages of macrophage differentiation and maturation in vivo. Third, the quantitative adherence of the mature macrophages after incubation with autologous lymphocytes and platelets allowed to plate macrophages at a reproducible cell density, which will increase the reproducibility of assays. Finally, fibronectin may maintain the functions of macrophages in vitro. Macrophages adhere preferentially to fibronectin-coated surfaces compared to laminin and other ECM components. Fibronectin has been recognized as the key element in promoting cell adhesion, spreading, various functions of macrophages. Therefore, our culture system provides a relatively stable model for studying macrophage behaviour.

Phagocytosis of Bacteria

Macrophages are phagocytes that can engulf microorganisms, tissue debris, and apoptotic cells, which perform protective and purified functions in healing. Phagocytosis is defined as the cellular engulfment of large particles, usually those over 0.5 μm in diameter. Phagocytosis is divided into three forms in virtue of dependent receptors. Type I phagocytosis, which is dependent on immunoglobulin receptors (FcyRs), induces actin-propelled extensions that surround the target particles, closing around in a zipper-like mechanism. At a molecular level, FcyRs triggering activates Rac and Cdc42-mediated cytoskeleton reorganization, such as actin polymerisation. Type II phagocytosis, which is dependent on complement receptors (e.g. CR3), occurs in the absence of actin-processed extension and involves particle sinking into the phagocytic cell membrane. However, actin polymerisation occurs at the contact area, which is dependent on the small GTPase RhoA but independent on Rac and Cdc42. Type III phagocytosis, which is dependent on phagocytic receptors, includes the engulfment of apoptotic bodies, which is crucial for the maintenance of cellular homeostasis. “Eat me” signals are recognized by phagocytic receptors that belong to different superfamilies, such as integrins (aᵥβ₃ and aᵥβ₅), lectins, and scavenger receptors.

Phagocytosis is generally summarized as the four steps, 1) chemotaxis—phagocytes are chemically attracted to the site of infection; 2) adherence—phagocyte plasma membrane attaches to the surface of pathogen or non-function cells; 3) ingestion—plasma membrane of phagocytes extends projections (pseudopods) to engulf and close pathogen in a sac, finally form phagosome; 4) digestion—inside the cell, phagosome fuses with lysosome to form phagolysosome. For example, the phagocytosis mediated by Fc receptor on the cell surface normally undergoes such an experience;

immunoglobulin molecules binding to Fc receptors→aggregation of

Fc receptors→local accumulation of tyrosine kinases→formation of F-actin nucleation sites→actin polymerisation and pseudopod extension. These steps continue in a cyclical fashion until particle engulfment is completed. In brief, phogocytosis is closely associated with actin polymerisation and GTPases (RhoA, Cdc42, and Rac) via FcyRs, complement receptors, or phagocytic receptors at the contact area. In our study, FITC-E. coli was used to study the phagocytosis of macrophages in the presence of 10% FCS. By quantitating the percentage of internalised cells, we found that the phagocytosis capacity was increased in a time-dependent manner in macrophages. It is in accordance with the character that macrophages are phagocytes. LIPUS increased the phagocytosis capacity, which accelerated the process of phagocytosis. Thus we consider that LIPUS is beneficial to speed the rate of healing. E.g. a bacterial infection.

Cytokine Synthesis

In healing, macrophages are not only immunological effector cells against invading environmental pathogens but also involved in inflammatory events and reparative processes through the production of pleitotropically active factors, among them, cytokines are prominent components. The main effects of cytokines are (a) recruitment and activation of pleitotropic cells, (b) regulation of pleitotropic cells proliferation and phagocytosis, (c) matrix remodelling (MMPs and ECM synthesis), (d) regulation of integrin and other cytokines expression, (e) angiogenesis and wound contraction, Therefore cytokines are vital modulators of healing, and they are in an exclusive position to integrate events and reparative processes.

Our findings showed LIPUS enhanced the expression of GM-CSF, IL-1 β, INF-y, MCP-3, TNF-a, EGF, VEGF, PDGF-β, FGF-9, IGFBP1, MIP-3a. Simultaneously, LIPUS decreased the expression of Osm and Tpo (FIG. 2). From these data we conclude that LIPUS regulated cytokines protein expression. Among the cytokines, VEGF, PDGFβ, IGF, EGF can promote cell proliferation and stimulate angiogenesis, chemokines (such as MCP-1, Gro-a, etc) contribute to attract inflammatory cells to the infected area. GM-CSF facilitates local recruitment of inflammatory cells and epithelial cells, and induces keratinocyte proliferation; therefore we suggest that LIPUS is beneficial to healing via the regulation of the cytokines.

In summary, the cytokines not only form a complex, interactive network, but also cooperate with ECM meshwork. It implies that the complex interplay among multiple cytokines, cells and ECM is central to the initiation, progression, and resolution of healing.

Matrix Remodelling

Matrix synthesis and degradation are vital for the reconstruction and remodelling of new tissues, which is necessary for normal healing. The MMPs constitute a family of endopeptidases that have a Zn²⁺ binding site. All these enzymes are secreted as proenzyme and, once activated, can degrade extracellular matrix components. By their proteolytic activity MMPs not only regulate matrix remodelling, but also promote the liberation of matrix-sequestered growth factors (e.g. TGF-β) and membrane bound proteins. In addition, MMps can cleave some cytokines (e.g. IGF, TGF-β, IL-1β) from their precursor form to an active form. Combining with the previous discussion on cytokines, we consider that, MMps affect the expression and function of cytokines, and certain cytokines induce MMps release. Therefore the interaction of MMPs and cytokines amplifies their functions in wound healing. Healing is delayed by inhibition of MMPs.

Different cells have their individual profiles of MMPs. Macrophages produce MMP-1, 2, 3, 7, and 9. MMP-9, the 92-kDa type IV collagenase(gelatinose) is the most prevalent form expressed by activated macrophages, which cleaves basement membrane collagen types IV and V, different types of gelatin, fibronectin, and elastin. Its proteolyctic activity is thought to be necessary for a variety of macrophage functions, such as cell migration and matrix remodelling.

Some literature reported that MMP-9 expression is increased by mechanical forces, for example, Magid and coworkers found that oscillatory shear stress increased secreted MMP-9 levels 2.7-fold over unidirectional shear stress in endothelial cells (Magid et al., 2003). Our results suggested that LIPUS increased MMP-9 expression and activity in adherent macrophages. Interestingly in suspended macrophages, no enhancement was observed by LIPUS treatment, which implies that cell adhesion is necessary for the effect induced LIPUS.

The precise mechanism how LIPUS affects the expression of MMPs in macrophages is still unclear. Current data suggest that the activities of MMPs can be controlled at three levels: (a) gene expression, (b) activation of the proenzyme forms of the MMPs (e.g. EMMPRIN and cytokines), (c) inhibition of specific inhibitors, such as tissue inhibitors of metalloproteinases (TIMPs). In our study, the effect of LIPUS on EMMPRIN and cytokines were detected, however, the direct effect of LIPUS and the regulation of TIMPs on MMPs are not presently examined.

Other names for EMMPRIN include basigin, M6, and CD147. EMMPRIN is a glycoprotein of 50-60 kDa having typical features of immunoglobulin superfamily, which contains two extracellular immunoglobulin domains, a transmembrane domain, and a 39amino acid cytoplasmic domain.

EMMPRIN is highly expressed on human peripheral blood cells and tumor cells, but its molecular function is still unclear. Some evidence implied the important role of EMMPRIN in tumor progression and metastasis by inducing MMPs synthesis. Lim reported that MMP-1, MMP-2, and MMP-3 are induced when exposed to EMMPRIN purified from tumor cells. EMMPRIN protein expression was upregulated by LIPUS treatment in adherent cells but not in suspended cells, which was similar to the effect of LIPUS on MMPs protein expression. Thus, the effect of LIPUS on MMPs synthesis can be at least partly induced via the upregulation of EMMPRIN expression.

Taken together, our data indicate that LIPUS accelerates phagocytosis, increases the protein expression of cytokines, MMPs, and EMMPRIN in adherent macrophages.

Potential Intracellular Reactions Induced by LIPUS.

Inititiation of Integrin-Medicated Signalling

Since LIPUS is a form of mechanical force, there should be some molecules acting as mechanosensors on cell surface to perceive the mechanical signals and transmit them into biochemical signals, finally influencing cellular reactions. Recent evidence suggest that integrins are one of the ideal mechanosensors. Integrins represent a complex family of cell adhesion receptors that bind to a variety of ECM ligands or cellular counter-receptors. Furthermore, the cytoplasmic domains of the integrins interact not only with actinbinding proteins, but also with focal adhesion tyrosine kinases related to a series of protein kinase cascades. Therefore integrins may transmit extracellular mechanical stimuli to the cells via cell-ECM contacts.

Cell adhesions represent the interactive sites between cells and ECM. At these sites, integrins bind to ECM ligands via their extracellular domains, and subsequently regulate cytoskeleton reorganization and focal contacts via their cytoplasmic domains. Cell-ECM contacts anchor elements of the ECM and the cytoskeleton, transmitting mechanical forces intracellularly, and initiating signalling events. Hereby cell-ECM contacts are key sites in integrin-mediated signalling events.

In a variety of cell types enhanced tyrosine phosphorylation of signalling proteins is a common response to integrin-mediated signal transduction. For example, mechanical stretch was shown to cause a rapid increase in tyrosine phosphorylation of some proteins, the molecular sizes were about 42, 44, 60, 70, 85, 120 and 170 kDa in cardiac myocytes. Schwartz and Short reported that activation of the MAPKs in anchored cells was far more effective than in cells maintained in suspension. Our results suggested that LIPUS treatment increased tyrosine phosphorylation of several components around 40-50 and 60-15-kDa in adherent macrophages and that adhesion via cell ECM contacts are required for integrin-mediated signalling events.

Deducted from the molecular weight of the bands in FIG. 9B, the possible proteins associated with LIPUS-induced signalling events are phosphatidylinositol 3-kinase (125 kDa), Syk (70 kDa), Src (60 kDa) and MAPKs (42 and 44 kDa). Thus we intend to give them brief introductions according to the literature on macrophages. P13K is involved in phagocytosis of macrophages by regulating membrane availability and local actin reorganization. PyK2 activation was implicated in several reactions like reorganization of the cytoskeleton, locomotion, and cell adhesion. Syk tyrosine phophorylation was closely correlated with N F_{K}B activation and the induction of immediate early genes, such as cytokines, that mediate the inflammatory response. Moreover, Syk activation was required for FcyR-mediated phagocytosis, actin assembly, and FcyRmediated transport to lyosomes.

The mass of data indicates that Src and MAPKs are important components for integrin-mediated signalling transducation, thereby we detected phosphorylation of Src and MAPKs in response to LIPUS in macrophages.

Activation of Src and p42/44 MAPK by LIPUS

Src family members are non-receptor protein tyrosine kinases. Macrophages contain five members of the Src family—Src, Hck, Fgr, Lyn and Fyn. Of these, Hck, Fgr, and Lyn are the predominant family members. The SH1 domain of Src constitutes the catalytic domain that includes the positive autophosphorylation site (tyr416) and the negative phosphorylation site (Tyr527). They participate in a variety of reactions, such as cytoskeletal assembly and organization, cell-matrix adhesion, induction of DNA synthesis, cell survival, and cellular proliferation. In macrophages, Src family kinases activation is considered to associate with phagocytosis and respiratory burst.

Our data together with Liu et al showed that mechanical forces increased Src activation. LIPUS increased Src phophorylation within 10 min after stimulation. Src phosphorylation peaked at 20 min in adherent macrophages.

MAPKs include more than a dozen proteins belonging to three families, p42/44 MAPK (extracellular signal-regulated kinases, ERKs), p38 MAPK, and c-Jun N-terminal kinase/stress-activated protein kinases (JNK/SAPK). P42/44 MAPK (ERK½) plays a critical role in the regulation of cell growth and differentiation. P38 MAPK participates in a signalling cascade controlling cellular responses to cytokines and stress. MAPKs can be activated in macrophages using a variety of stimuli. In our system, LIPUS increased the phosphorylation of p42/44 MAPK, which implies that p42/44 MAPK are involved in LIPUS-induced signalling events in macrophages.

The importance of MAPKs has been established in mammalian cell biology in numerous studies using a wide variety of model systems. MAPKs activation is involved in cell proliferation, differentiation, and regulation of proinflammatory mediators. Recent investigation showed the role of MAPK pathway in the expression of MMPs. Macrophages require p42/44 MAPKs for efficient FcyR-mediated phagocytosis. We show that p42/44 MAPK may be involved in the increase of cytokines, EMMPRIN and MMPs expression, and speed up the phagocytosis.

Formation of Focal Complexes

Cell-ECM adhesion sites are termed focal contacts or focal adhesions. There are two forms in macrophages, focal complexes and podosomes. Focal complexes are small, dot-like adhesions present at the edges of lamellipodia. Podosome are small (˜0.5 μm) cylindrical structures containing an actin core surrounded by tyrosine-phosphorylated proteins and several typical focal contact proteins, such as vinculin and talin.

Unlike fibroblasts, macrophages do not form large, well-organized focal contacts with attached stress fibers. Instead, they maintain the membrane skeleton in a more dynamic state, with poorly organized focal complexes from which fine actin cables infrequently arise. This might explain their higher motility rates compared with fibroblasts or endothelial cells. We found that macrophages presented punctuate F-actin structures that diffusely distributed in the cytoplasm or locally distributed in the protrusion of cells, dot-like tyrosine phosphorylated proteins accumulating along the cell margin, and few fine actin cables in cultured macrophages stimulated with LIPUS for 10 min. Jones reported that only in the presence of CSF-1, macrophages from obvious actin cables that parallel the polarized axis of the cell or circumferentially round the edge of the cell.

When cells were challenged by LIPUS, they presented more focal complexes and tyrosine-phosphorylated proteins, which illustrates that LIPUS induces the formation and tyrosine phosphorylation of focal complexes. Thus, we consider that focal complexes are involved in LIPUS-induced signal transduction.

In summary, we proved that LIPUS accelerates human macrophages phagocytosis, increases the expression of several cytokines, and regulates matrix remodelling. In addition, LIPUS increases tyrosine phosphorylation, activates Src and ERK, and induces the formation of focal complexes. This suggests, that mechanical signals produced by LIPUS can be transmitted to the intracellular compartments at cell-ECM contacts, subsequently mediating a series of signalling events.

LIPUS accelerated phagocytosis, and stimulated the synthesis and release of the cytokines, GM-CSF, 1-309, IL-1 β, INF-y, MCP-3. TNFa, EGF, VEGF, PDGF-β, FGF-9, IGFBP-1, and MIP-3a in cultured adherent macrophages. The protein expression of MMP-9 as well as cell membrane-associated EMMPRIN increased gradually in response to 10, 20, 30, and 40 min LIPUS, showing a peak with 40 min stimulation in adherent macrophages. Soluble EMMPRIN was also increased by LIPUS. However, when cells were in suspension, LIPUS had no effect on MMP-9 and EMMPRIN protein expression.

Furthermore, 10 min LIPUS enhanced tyrosine phosphorylation of several proteins in adherent macrophages, but not in suspended cells. Using phospho-specific antibodies we demonstrated that LIPUS activated Src and ERK, but not p38 MAPK in adherent macrophages. The activation of Src and ERK was increased 10 to 20 min and 20 to 40 min after stimulation, respectively. In addition, LIPUS induced F-actin polymerisation and the formation of focal complexes.

Our data shows that mechanical signals of LIPUS are transmitted into cells via cell-extracellular matrix (ECM) contacts, and trigger signalling events, such as F-actin polymerisation, the formation of focal complexes, and the activation of Src and ERK. As a consequence of LIPUS application to macrophages phagocytosis is accelerated, and the expression of cytokines, MMPs, and EMMPRIN is stimulated.

1. A method for treating a bacterial and/or fungal infection comprising applying ultrasound to, adjacent, or near an infected site.

2. The method of claim 1 wherein applying ultrasound comprises applying a pulsed radio frequency ultrasound signal.

3. The method of claim 2 wherein the pulsed radio frequency signal has a frequency in the range of 1.3-2 Mhz and comprises pulses generated at a rate in the range 100-1000 mHz, with each pulse having a duration in the range 10-2000 microseconds.

4. The method of claim 1, wherein applying ultrasound comprises applying an ultrasound signal, wherein a power intensity of the ultrasound signal is no higher than 100 milliwatts per square centimeter.

5. The method of claim 1, wherein applying ultrasound comprises applying ultrasound daily, for at least thirty days, for only a small part of each day.

6. The method of claim 1, wherein applying ultrasound comprises applying ultrasound daily, for at least twenty days, for only a small part of each day.

7. The method of claim 1, wherein applying ultrasound comprises directing ultrasound to a cellular mileau of the infected site.

8. The method of claim 1, wherein applying ultrasound comprises directing ultrasound at one or more phagocytic cells.

9. The method of claim 1, wherein applying ultrasound comprises directing ultrasound at one or more hematopoietic cells of a bone marrow lineage.

10. The method of claim 1, wherein applying ultrasound comprises directing ultrasound at one or more macrophage cells.

11. The method of claim 1, wherein applying ultrasound comprises directing ultrasound at a tissue of the body.

12. The method of claim 1, wherein applying ultrasound comprises applying ultrasound to treat osteomyelitis.

13. The method of claim 1, wherein applying ultrasound comprises applying ultrasound to treat meningitis.

14. The method of claim 1, wherein applying ultrasound comprises applying ultrasound to treat candidiasis.

15. The method of claim 1, wherein applying ultrasound comprises applying ultrasound to treat cellulutis.

16. An ultrasound emitting device which emits an ultrasound signal capable of treating a bacterial and/or fungal infection.

17. A method for cytokine production and/or enhancing activity of cytokine comprising applying ultrasound to a cell, cells and/or tissue.

18. A method of MMP production and/or enchancing activity of MMP comprising applying ultrasound to a cell, cells and/or tissue.

19. A method of EMMPRIN production and/or enhancing activity of EMMPRIN comprising applying ultrasound to a cell, cells and/or tissue.
